(19) United States
(12) Patent Application Publication
(10) Pub. No.: US 2009/0246805 A1
(43) Pub. Date: Oct. 1, 2009

(54) METHOD FOR CLASSIFYING AND COUNTING BASOPHILS

(75) Inventors: Keiko Moriyama, Kobe-shi (JP); Yuji Itose, Kobe-shi (JP); Tatsuya Narikawa, Kobe-shi (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION, Kobe-shi (JP)

(21) Appl. No.: 12/402,205

(22) Filed: Mar. 11, 2009

(30) Foreign Application Priority Data
Mar. 28, 2008 (JP) 2008-085231

(51) Int. Cl. G01N 33/567

(52) U.S. Cl. 435/7.24; 435/7.21

(57) ABSTRACT

A method for counting basophils which comprises: (1) mixing and reacting a blood sample with an anti-CD123 antibody labeled with a first fluorescent label and an anti-CD294 antibody labeled with a second fluorescent label to prepare a measurement sample, (2) introducing the measurement sample into a flow cell of a flow cytometer and irradiating, with light, cells in the measurement sample flowing in the flow cell, (3) detecting fluorescences from the first and second fluorescent labels as well as two scattered lights different in angle, emitted from the cells, (4) identifying basophils on the basis of the detected fluorescences from the first and second fluorescent labels as well as the two scattered lights different in angle, and (5) counting the identified basophils. A kit for measuring basophils which comprises a CD123 antibody labeled with a first fluorescent label and a CD294 antibody labeled with a second fluorescent label.

### FIELD OF THE INVENTION

The present invention relates to a method for classifying and counting basophils by using flow cytometry method.

### BACKGROUND

The proportion of basophils in leukocytes is as low as 0 to 2% in human whole blood, and counting by a visual observation method is sensitive and specific, but is problematic in reproducibility and difficult to accurately calculate basophils. In recent years, monoclonal antibodies reacting with surface antigens (surface markers) expressed on the membrane surfaces of leukocytes are used in cell analysis by flow cytometry. The method of measuring basophils by flow cytometry includes, for example, a method of using a CD123 antibody and an HLA-DR antibody to fractionate CD123 (anti-IL-3Rα)-positive and HLA-DR-negative cells as basophils and a method of using a CD123 antibody and a CD303 antibody to fractionate CD123-positive and CD303-negative cells as basophils. These methods identify basophils with only CD123and are thus poor in specificity. Other methods include a method of using a CD123 antibody and a CD203c antibody to fractionate CD123-positive and CD203c-positive cells as activated basophils and a method of using a CD294 antibody, a CD203c antibody and a CD3 antibody to fractionate CD294-positive and CD203c-positive cells as basophils. However, CD203c detected in these methods is low in expression level when basophils are not activated, and thus these methods are unsuitable when basophils are to be quantified irrespectively of activation level.

As examples of measurement of basophils by flow cytometry, there are JP-B 8-1434, JP-T 2002-525580 and JP-T 2004-533855 although not specialized in measurement of basophils only. In JP-B 8-1434, a blood sample is treated with a CD45 antibody, a CD71 antibody and thiazole orange, and then 3 fluorescences and 2 scattered lights are detected to analyze blood cells. In JP-T 2002-525580, eosinophils and basophils are quantified with a combination of an IL-5 receptor antibody, a CD3 antibody, a CD16 antibody and a CD19 antibody. In JP-T 2004-533855, leukocytes are classified into 5 groups with a combination of a CD4 antibody and a CD45 antibody.

### SUMMARY

The scope of the present invention is defined solely by the appended claims, and is not affected to any degree by the statements within this summary.

A first aspect of the present invention is a method for counting basophils which comprises:
(1) mixing and reacting a blood sample with an anti-CD123 antibody labeled with a first fluorescent label and an anti-CD294 antibody labeled with a second fluorescent label to prepare a measurement sample,
(2) introducing the measurement sample into a flow cell of a flow cytometer and irradiating, with light, cells in the measurement sample flowing in the flow cell,
(3) detecting fluorescences from the first and second fluorescent labels as well as two scattered lights different in angle, emitted from the cells,
(4) identifying basophils on the basis of the detected fluorescences from the first and second fluorescent labels as well as the two scattered lights different in angle, and
(5) counting the identified basophils.

A second aspect of the present invention is a method for counting basophils which comprises:
(1) mixing and reacting a blood sample with an anti-CD123 antibody labeled with a first fluorescent label, an anti-CD294 antibody labeled with a second fluorescent label and an anti-CD45 antibody labeled with a third fluorescent label to prepare a measurement sample,
(2) introducing the measurement sample into a flow cell of a flow cytometer and irradiating, with light, cells in the measurement sample flowing in the flow cell,
(3) detecting fluorescences from the first, second and third fluorescent labels as well as side scattered light, emitted from the cells,
(4) identifying basophils on the basis of the detected fluorescences from the first, second and third fluorescent labels as well as the scattered light, and
(5) counting the identified basophils.

A third aspect of the present invention is a kit for measuring basophils which comprises a CD123 antibody labeled with a first fluorescent label, and a CD294 antibody labeled with a second fluorescent label.

### BRIEF DESCRIPTION OF THE DRAWINGS

FIG. 1 is an example of the optical system of a flow cytometer used in the present invention;

FIG. 2 is a CD45 fluorescence intensity/side scattered light intensity scattergram in Example 1;

FIG. 3 is a forward scattered light intensity/side scattered light intensity scattergram in Example 1;

FIG. 4 is a CD294-PE/CD123-FITC scattergram of cells contained in two monocyte areas in Example 1;

FIG. 5 is a CD294-PE/CD123-FITC scattergram of cells contained in P2 area in Example 1;

FIG. 6 is a CD294-PE/CD123-FITC scattergram of cells contained in P3 area in Example 1;

FIG. 7 is a CD45 fluorescence intensity/side scattered light intensity scattergram in Example 2;

FIG. 8 is a forward scattered light intensity/side scattered light intensity scattergram in Example 2;

FIG. 9 is a CD294-PE/CD123-FITC scattergram of cells contained in two monocyte areas in Example 2;

FIG. 10 is a CD294-PE/CD123-FITC scattergram of cells contained in P2 area in Example 2;

FIG. 11 is a CD294-PE/CD123-FITC scattergram of cells contained in P3 area in Example 2;

FIG. 12 is a diagram of correlation with a visual observation method in Example 3; and

FIG. 13 shows an example of the basophil measurement kit of the present invention.

### DETAILED DESCRIPTION OF THE PREFERRED EMBODIMENTS

The preferred embodiments of the present invention are described hereinafter with reference to the drawings.

The anti-CD45 antibody used in the present invention reacts with all leukocytes. On the membrane surfaces of all leukocytes, CD45 antigens are expressed at varying degrees depending on the type of leukocyte. Accordingly, this antibody can be used in fractionation and quantification of leukocytes.

The anti-CD123 antibody used in the present invention reacts with interleukin receptor-3α chains expressed on peripheral blood dendritic cells, precursor cells, monocytes, eosinophils and basophils.

The anti-CD294 antibody used in the present invention binds to CRTH2 known as prostaglandin D2 receptor. CRTH2 is a marker of inflammatory cells including basophils. In healthy human whole blood, CRTH2 is expressed on T-helper 2 cells, cytotoxic T cells, eosinophils and basophils involved in immune responses and allergic reactions.

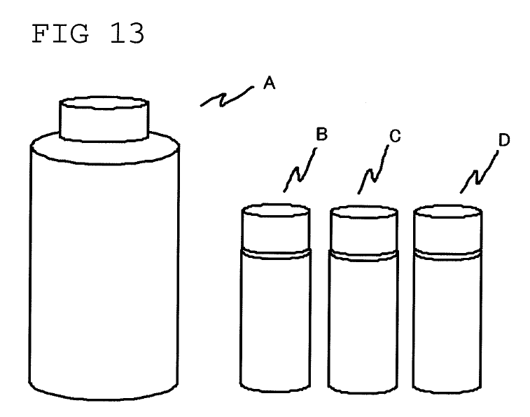

The antibodies used in the present invention are labeled with fluorescent dyes distinguishable from one another. Examples of the fluorescent dyes used as the label include peridinin chlorophyll complex (PerCP), fluorescein isothiocyanate (FITC), phycoerythrin (PE), allophycocyanin (APC), Texas Red (TR) and CY5. Among them, those distinguishable from one another can be appropriately selected and used as the label. The antibodies used in the present invention may be commercial products. The antibodies used in the present invention may be combined with a hemolyzing agent described later to constitute “a basophil measurement kit”. An example of the kit is shown in FIG. 13. In FIG. 13, the reference character A denotes a hemolyzing agent, the reference character B denotes a CD123 antibody reagent, the reference character C denotes a CD294 antibody reagent, and the reference character D denotes a CD45 antibody reagent. The kit in FIG. 13 contains all of the reference characters A to D, but only the reference characters A to C, only the reference characters B to D, or only the reference characters B and C may be used to constitute the “basophil measurement kit”.

The scattered light as used herein refers to light whose traveling direction is changed by particles such as blood cells present in the traveling direction of the light. The scattered light includes, for example, forward scattered light and side scattered light. The forward scattered light refers to scattered light which is emitted from particles at approximately the same angle as the traveling direction of light applied from a light source. The side scattered light refers to scattered light which is emitted from particles at an angle of approximately 90° C. relative to the traveling direction of light applied from a light source. Two scattered lights different in angle are not particularly limited as long as the scattered lights are those emitted from particles at angles different from each other relative to the traveling direction of light applied from a light source. Preferable examples of the two lights different in angle are forward scattered light and side scattered light.

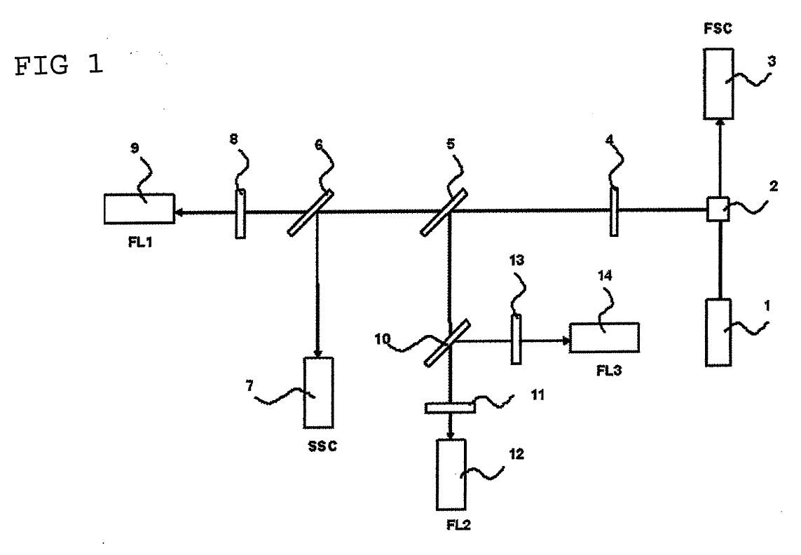

An example of the optical system of a flow cytometer used in the present invention is shown in FIG. 1. Light that has been emitted from a light source 1 (which, in Examples of the present invention, is an argon laser) is introduced into an orifice portion in a sheath flow cell 2.

Forward scattered light that is emitted from cells, which are discharged from a nozzle (not shown) and pass through the orifice portion, enters into a forward scattered light detector (FSC) 3.

Meanwhile, side scattered light that is emitted from cells passing through the orifice portion enters into a side scattered light detector (SSC) 7 via a condenser lens 4, a dichroic mirror 5 and a beam splitter 6. In the side fluorescence that is emitted from the cells passing through the orifice portion, fluorescence of shortest fluorescence wavelength (which, in Examples in this specification, is FITC fluorescence) enters into a side fluorescence detector (FL1) (photomultiplier tube) 9 via condenser lens 4, dichroic mirror 5, beam splitter 6 and a filter 8. Fluorescence of second shortest fluorescence wavelength (which, in Examples in this specification, is PE fluorescence) enters into aside fluorescence detector (FL2) (photomultiplier tube) 12 via condenser lens 4, dichroic mirror 5, a dichroic mirror 10 and a filter 11. Fluorescence of longest fluorescence wavelength (which, in Examples in this specification, is PerCP fluorescence) enters into a side fluorescence detector (FL3) (photomultiplier tube) 14 via condenser lens 4, dichroic mirror 5, dichroic mirror 10 and a filter 13.

A forward scattered light signal outputted from the forward scattered light detector 3, a side scattered light signal outputted from the side scattered light detector 7, and side fluorescence signals outputted from the side fluorescence detector (FL1) 9, the side fluorescence detector (FL2) 12 and the side fluorescence detector (FL3) 14, respectively, are amplified by an amplifier (not shown) and inputted into analyzing part (not shown).

The analyzing part performs predetermined analysis and a desired arithmetical operation and allows calculation results and operation results to be displayed on a display (not shown).

The method for classifying and counting basophils according to the present invention is carried out in the following manner. First, a blood sample is reacted with the respective fluorescently labeled antibodies to prepare a measurement sample. A measurement sample may also be prepared by adding a hemolyzing agent if necessary to lyse erythrocytes in the blood sample, then centrifuging the sample to remove a supernatant and re-suspending the precipitates in a buffer.

As the blood sample, a peripheral blood or bone marrow blood sample can be used.

As the hemolyzing agent, a commercial hemolyzing agent can be used. For example, a hemolyzing agent based on ammonium chloride is preferably used.

Then, the measurement sample prepared above is introduced into a flow cell of a flow cytometer; cells in the measurement sample which flow in the flow cell are irradiated with light; fluorescent label-derived fluorescences and scattered lights emitted from the cells are detected; and basophils are identified on the basis of the detected fluorescent label-derived fluorescences and scattered lights.

The method of identifying basophils includes (1) a method of analysis with a forward scattered light intensity/side scattered light intensity scattergram and a CD45 fluorescence intensity/side scattered light intensity scattergram, (2) a method of analysis with a CD45 fluorescence intensity/side scattered light intensity scattergram, and (3) a method of analysis with a forward scattered light intensity/side scattered light intensity scattergram.

First, the method of analysis with a forward scattered light intensity/side scattered light intensity scattergram and a CD45 fluorescence intensity/side scattered light intensity scattergram (the method (1)) is described.

On the basis of the respective signals detected by measurement, a scattergram with forward scattered light intensity and side scattered light intensity as two axes (forward scattered light intensity/side scattered light intensity scattergram) and a scattergram with CD45 fluorescence intensity (fluorescence intensity from an anti-CD45 fluorescently labeled antibody bound to cells) and side scattered light intensity as two axes (CD45 fluorescence intensity/side scattered light intensity scattergram) are prepared. A monocyte region (P2 area) containing lymphocytes, monocytes and basophils is specified on the CD45 fluorescence intensity/side scattered light intensity scattergram. A monocyte region (P3 area) containing lymphocytes, monocytes and basophils is specified on the forward scattered light intensity/side scattered light intensity scattergram.

For the cells appearing in both the P2 and P3 areas, a scattergram with CD123 fluorescence intensity (fluorescence intensity from an anti-CD123 fluorescently labeled antibody bound to cells) and CD294 fluorescence intensity (fluorescence intensity from an anti-CD294 fluorescently labeled antibody bound to cells) as two axes (CD123 fluorescence intensity/CD294 fluorescence intensity scattergram) is then prepared. A region containing CD294-positive and CD123-positive cells is specified (BASO area) on the CD123 fluorescence intensity/CD294 fluorescence intensity scattergram, and the cells in the BASO area are counted to determine the number of basophils. A region containing whole leukocytes is specified (WBC area) on the CD45 fluorescence intensity/side scattered light intensity scattergram, and the cells in the WBC area are counted to determine the number of leukocytes, whereby the proportion of basophils can be calculated.

Then, the method of analysis with a CD45 fluorescence intensity/side scattered light intensity scattergram (the method (2)) is described.

On the basis of the respective signals detected by measurement, a scattergram with CD45 fluorescence intensity (fluorescence intensity from an anti-CD45 fluorescently labeled antibody bound to cells) and side scattered light intensity as two axes (CD45 fluorescence intensity/side scattered light intensity scattergram) is prepared. A monocyte region (P2 area) containing lymphocytes, monocytes and basophils is specified on the CD45 fluorescence intensity/side scattered light intensity scattergram.

For the cells appearing in the P2 area, a scattergram with CD123 fluorescence intensity (fluorescence intensity from an anti-CD123 fluorescently labeled antibody bound to cells) and CD294 fluorescence intensity (fluorescence intensity from an anti-CD294 fluorescently labeled antibody bound to cells) as two axes (CD123 fluorescence intensity/CD294 fluorescence intensity scattergram) is then prepared. A region containing CD294-positive and CD123-positive cells is specified (P2-BASO area) on the CD123 fluorescence intensity/CD294 fluorescence intensity scattergram, and the cells in the P2-BASO area are counted to determine the number of basophils. A region containing whole leukocytes is specified (WBC area) on the CD45 fluorescence intensity/side scattered light intensity scattergram, and the cells in the WBC area are counted to determine the number of leukocytes, whereby the proportion of basophils can be calculated.

Then, the method of analysis with a forward scattered light intensity/side scattered light intensity scattergram (the method (3)) is described.

On the basis of the respective signals detected by measurement, a forward scattered light intensity/side scattered light intensity scattergram is prepared. A monocyte region (P3 area) containing lymphocytes, monocytes and basophils is specified on the forward scattered light intensity/side scattered light intensity scattergram.

For the cells appearing in the P3 area, a CD123 fluorescence intensity/CD294 fluorescence intensity scattergram is then prepared. A region containing CD294-positive and CD123-positive cells is specified (P3-BASO area) on the CD123 fluorescence intensity/CD294 fluorescence intensity scattergram, and the cells in the P3-BASO area are counted to determine the number of basophils. A region containing whole leukocytes is specified (WBC2 area) on the forward scattered light intensity/side scattered light intensity scattergram, and the cells in the WBC2 area are counted to determine the number of leukocytes, whereby the proportion of basophils can be calculated.

For analysis by the method (3), the anti-CD45 labeled antibody is not always required but can be used when the number of whole leukocytes is to be determined.

In the method of identifying basophils as described above, a monocyte region containing basophils is specified, and then BASO area is specified by CD123 fluorescence intensity and CD294 fluorescence intensity, thereby counting the number of basophils; however, the method of the present invention is not limited thereto. For example, a region containing basophils is specified by CD123 fluorescence intensity and CD294 fluorescence intensity, and BASO area in the specified region is further specified by forward scattered light intensity and side scattered light intensity, thereby counting the number of basophils. Alternatively, a region containing basophils is specified by CD123 fluorescence intensity and CD294 fluorescence intensity, and BASO area in the specified region is further specified by CD45 fluorescence intensity and side scattered light intensity, thereby counting the number of basophils. Alternatively, a region containing basophils is specified by CD123 fluorescence intensity and CD294 fluorescence intensity, and BASO area in the specified region is further specified by forward scattered light intensity and side scattered light intensity and by CD45 fluorescence intensity and side scattered light intensity, thereby counting the number of basophils. Alternatively, regions containing basophils are specified respectively by CD123 fluorescence intensity and CD294 fluorescence intensity and by forward scattered light intensity and side scattered light intensity, and cells appearing commonly in both the specified regions can be counted as basophils. Alternatively, regions containing basophils are specified respectively by CD123 fluorescence intensity and CD294 fluorescence intensity and by CD45 fluorescence intensity and side scattered light intensity, and cells appearing commonly in both the specified regions can be counted as basophils. Alternatively, regions containing basophils are obtained respectively by CD123 fluorescence intensity and CD294 fluorescence intensity, by forward scattered light intensity and side scattered light intensity and by CD45 fluorescence intensity and side scattered light intensity, and cells appearing commonly in all the obtained regions can be counted as basophils.

In the present invention, the two antibodies CD123 and CD294 are used to detect basophils irrespectively of their activation level. Detection of basophils by CD123 and CD294 does not need any specific antibody for removal of CD123-positive dendritic cells. Without using another specific antibody, false-positive results attributable to eosinophils or the like can be circumvented by a gating strategy in which a monocyte area is gated on a forward scattered light intensity/side scattered light intensity scattergram and/or a CD45 fluorescent intensity/side scattered light intensity scattergram, and CD123-positive and CD294-positive cells out of cells contained in the monocyte area are gated as basophils.

According to the present invention, basophils can be clearly fractionated from other leukocytes, and basophils can be accurately counted.

### EXAMPLES

### Example 1

Five μl each of a PerCP-labeled anti-CD45 antibody, a FITC-labeled anti-CD123 antibody and a PE-labeled anti-CD294 antibody are added to 50 μl of an anticoagulant-containing peripheral blood sample and incubated for 15 minutes at room temperature in a dark place. Two ml of a hemolyzing agent based on ammonium chloride is added to the sample and incubated for about 15 minutes at room temperature in a dark place.

After hemolysis treatment, the sample is centrifuged at 1000 rpm for 5 minutes, then a supernatant is removed, and the remaining pellet is re-suspended in 1 ml PBS. Then, the sample is measured with a flow cytometer (FACS Canto manufactured by Becton Dickinson). 30,000 counts are measured per sample.

(1) Analysis with a Forward Scattered Light Intensity/Side Scattered Light Intensity Scattergram and a CD45 Fluorescence Intensity/Side Scattered Light Intensity Scattergram

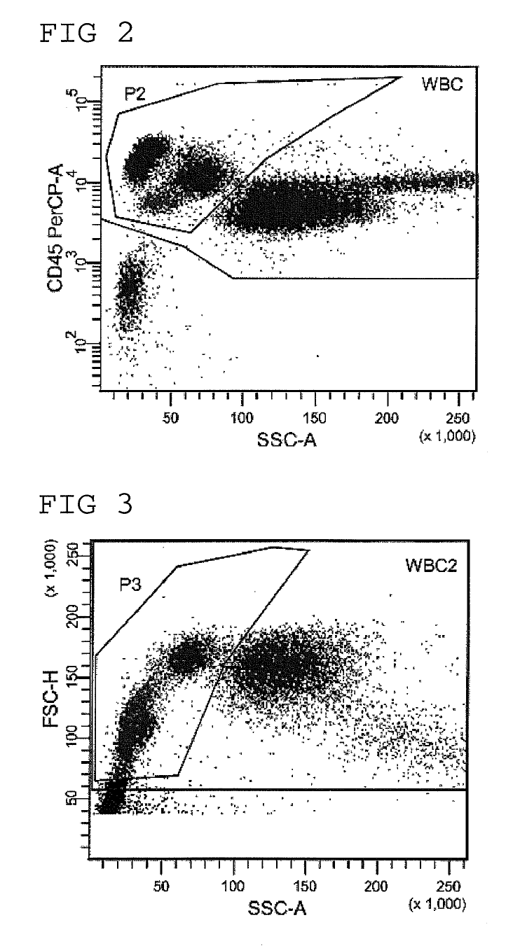

The whole cells are displayed on a forward scattered light intensity (FSC)/side scattered light intensity (SSC) scattergram (FIG. 3) and on a CD45-PerCP/SSC scattergram (FIG. 2).

In both the scattergrams, monocyte areas excluding neutrophils, eosinophils, and erythrocyte ghosts are gated (P2 and P3 areas).

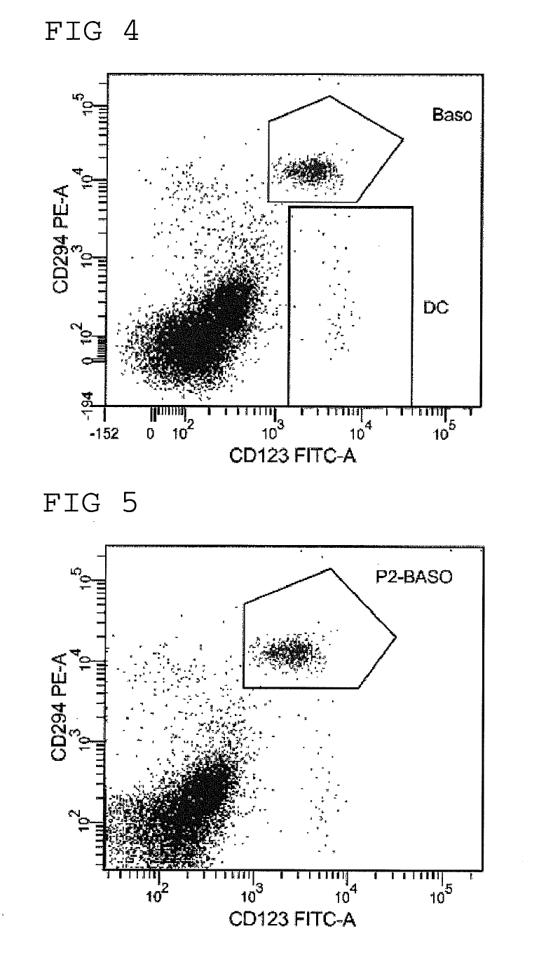

Cells contained commonly in both the monocyte areas are displayed on a CD294-PE/CD123-FITC scattergram (FIG. 4).

CD294-positive and CD123-positive (BASO area) cells on this scattergram are counted to determine the number of basophils. On the scattergram in FIG. 2, a region containing whole erythrocytes is specified (WBC area), cells in the WBC area are counted to determine the number of leukocytes, and the proportion of basophils is calculated.

It was found that basophils could be clearly identified.

(2) Analysis with a CD45 Fluorescence Intensity/Side Scattered Light Intensity Scattergram

On the scattergram in FIG. 2, a monocyte area excluding neutrophils, eosinophils, and erythrocyte ghosts is gated (P2 area).

Cells contained in the P2 area are displayed on a CD294-PE/CD123-FITC scattergram (FIG. 5).

CD294-positive and CD123-positive (P2-BASO area) cells on this scattergram are counted to determine the number of basophils. From the scattergram in FIG. 2, the number of leukocytes is determined, and the proportion of basophils is calculated.

Basophils could be identified without using a forward scattered light intensity/side scattered light intensity scattergram.

(3) Analysis with a Forward Scattered Light Intensity/Side Scattered Light Intensity Scattergram

On the scattergram in FIG. 3, a monocyte area excluding neutrophils, eosinophils and erythrocyte ghosts is gated (P3 area).

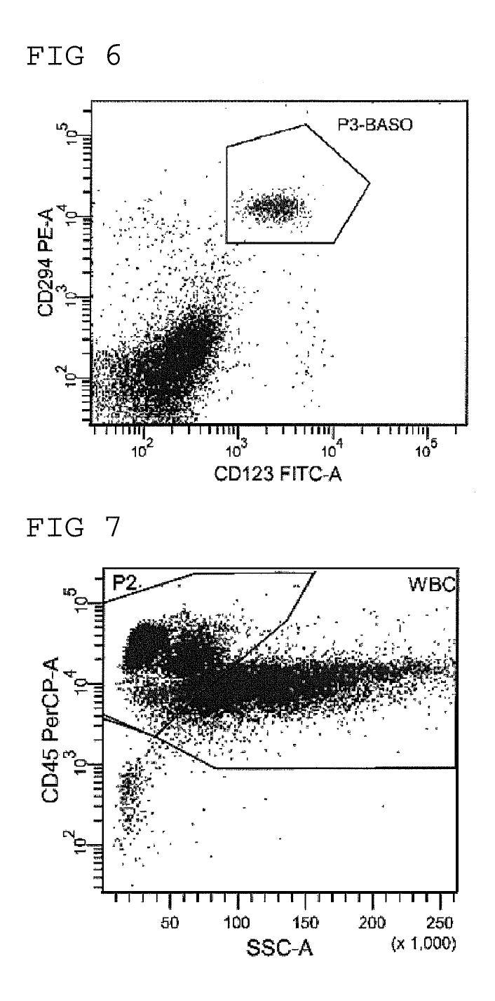

Cells contained in the P3 area are displayed on a CD294-PE/CD123-FITC scattergram (FIG. 6).

CD294-positive and CD123-positive (P3-BASO area) cells on this scattergram are counted to determine the number of basophils. On the scattergram in FIG. 3, a region containing whole leukocytes is specified (WBC2 area). Cells in the WBC2 area are contented to determine the number of leukocytes, and the proportion of basophils is calculated.

Basophils could be identified without using a CD45 fluorescence intensity/side scattered light intensity scattergram.

### Example 2

### Measurement of Basophils in a Sample Poor in Fractionation

A sample poor in leukocyte fractionation was measured in the same manner as in Example 1. The sample contained lymphocytes, monocytes and basophils, but those cells in the sample were hardly differentiated from each other by a traditional method.

(1) Analysis with a Forward Scattered Light Intensity/Side Scattered Light Intensity Scattergram and a CD45 Fluorescence Intensity/Side Scattered Light Intensity Scattergram

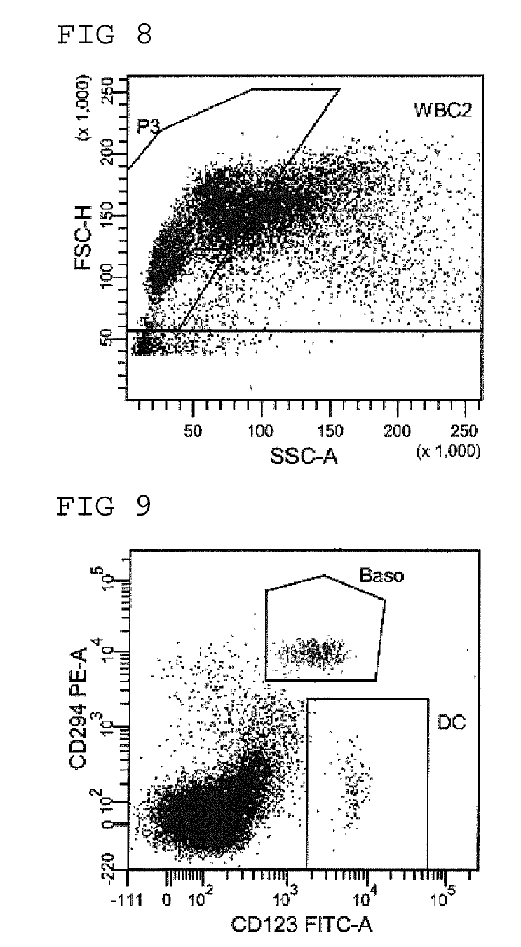

The same method as in Example 1 was used in analysis. A CD45 fluorescence intensity/side scattered light intensity scattergram is shown in FIG. 7. A forward scattered light intensity/side scattered light intensity scattergram is shown in FIG. 8. A CD294-PE/CD123-FITC scattergram is shown in FIG. 9. It was found that even in a sample poor in leukocyte fractionation, basophils could be clearly identified.

(2) Analysis with a CD45 Fluorescence Intensity/Side Scattered Light Intensity Scattergram

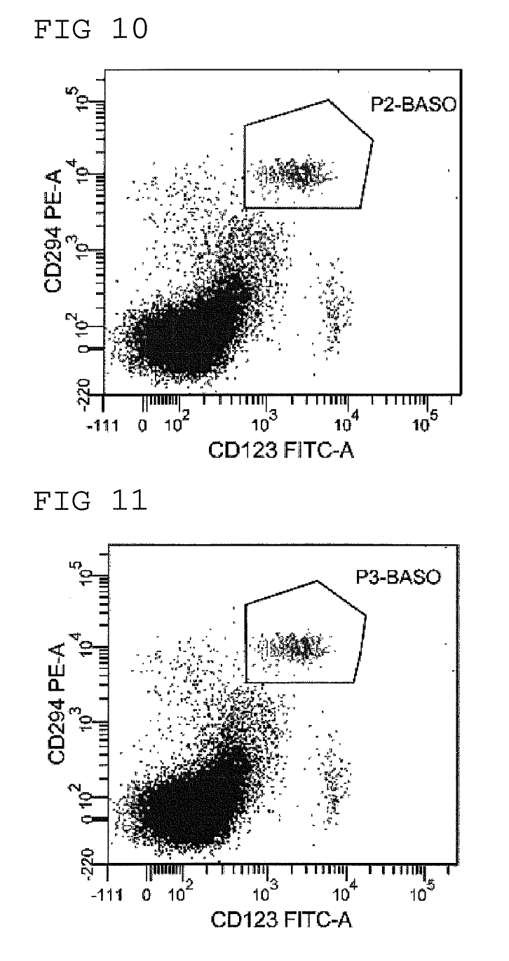

The same method as in Example 1 was used in analysis. A CD294-PE/CD123-FITC scattergram is shown in FIG. 10.

Even in a sample poor in leukocyte fractionation, basophils can be clearly identified without using a forward scattered light intensity/side scattered light intensity scattergram.

(3) Analysis with a Forward Scattered Light Intensity/Side Scattered Light Intensity Scattergram

The same method as in Example 1 was used in analysis. A CD294-PE/CD123-FITC scattergram is shown in FIG. 11.

Even in a sample poor in leukocyte fractionation, basophils can be clearly identified without using a forward scattered light intensity/side scattered light intensity scattergram.

### Example 3

Correlation with a Visual Observation Method

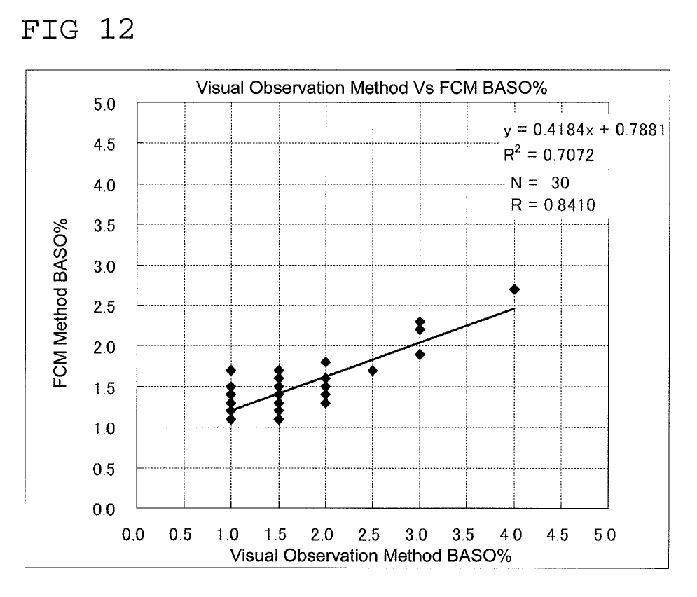

Thirty clinical samples were measured in the same manner as in Example 1 and analyzed in the same manner as in (1) in Example 1, to examine correlation with a visual observation method (100 counts). The results are shown in Table 1, and a correlation diagram is shown in FIG. 12.

The visual observation method (visual observation method BASO %) and the method of the present invention (FCM method BASO %) gave almost the same results, thus showing good correlation of the present method with the visual observation method.

TABLE 1

| No. | Visual observation method BASO % | FCM BASO % |
|---|---|---|
| 1 | 4.0 | 2.7 |
| 2 | 2.5 | 1.7 |
| 3 | 2.0 | 1.5 |
| 4 | 1.0 | 1.3 |
| 5 | 3.0 | 1.9 |
| 6 | 3.0 | 2.2 |
| 7 | 1.5 | 1.3 |
| 8 | 1.0 | 1.5 |
| 9 | 1.5 | 1.6 |
| 10 | 2.0 | 1.8 |
| 11 | 2.0 | 1.6 |
| 12 | 1.0 | 1.4 |
| 13 | 1.5 | 1.2 |
| 14 | 2.0 | 1.5 |
| 15 | 1.0 | 1.2 |
| 16 | 2.0 | 1.6 |
| 17 | 2.0 | 1.3 |
| 18 | 1.5 | 1.5 |
| 19 | 3.0 | 2.3 |
| 20 | 1.5 | 1.7 |
| 21 | 1.0 | 1.1 |
| 22 | 1.0 | 1.1 |
| 23 | 1.0 | 1.7 |
| 24 | 2.0 | 1.4 |
| 25 | 1.5 | 1.1 |
| 26 | 1.0 | 1.3 |
| 27 | 2.0 | 1.3 |
| 28 | 1.0 | 1.1 |
| 29 | 1.5 | 1.4 |
| 30 | 1.0 | 1.1 |

The present invention is useful in accurately counting basophils in clinical examination.

What is claimed is:

1. A method for counting basophils which comprises:
(1) mixing and reacting a blood sample with an anti-CD123 antibody labeled with a first fluorescent label and an anti-CD294 antibody labeled with a second fluorescent label to prepare a measurement sample,
(2) introducing the measurement sample into a flow cell of a flow cytometer and irradiating, with light, cells in the measurement sample flowing in the flow cell,
(3) detecting fluorescences from the first and second fluorescent labels as well as two scattered lights different in angle, emitted from the cells,
(4) identifying basophils on the basis of the detected fluorescences from the first and second fluorescent labels as well as the two scattered lights different in angle, and
(5) counting the identified basophils.

2. The method according to claim 1, wherein the two scattered lights different in angle are forward scattered light and side scattered light.

3. The method according to claim 1, wherein the step (4) comprises:
specifying a cell population containing lymphocytes, monocytes and basophils contained in the blood sample, on the basis of the two scattered lights different in angle, and
identifying basophils in cells contained in the specified cell population, on the basis of the fluorescences from the first and second fluorescent labels.

4. The method according to claim 1, wherein the step (1) comprises treating, with a hemolyzing agent, the blood sample mixed and reacted with the antibodies, thereby lysing erythrocytes in the blood sample.

5. The method according to claim 4, wherein the hemolyzing agent is based on ammonium chloride.

6. The method according to claim 1, wherein the first and second fluorescent labels are fluorescent labels different from each other, which are selected from the group consisting of peridinin chlorophyll complex, fluorescein isothiocyanate, phycoerythrin, allophycocyanin, Texas Red and CY5.

7. The method according to claim 1, wherein:
the step (1) further comprises mixing an anti-CD45 antibody labeled with a third fluorescent label,
the step (3) further comprises detecting fluorescence from the third fluorescent label, and
the step (4) comprises identifying basophils on the basis of the detected fluorescences from the first, second and third fluorescent labels as well as the two scattered lights different in angle.

8. The method according to claim 7, wherein the two scattered lights different in angle are forward scattered light and side scattered light.

9. The method according to claim 8, wherein the step (4) comprises:
specifying a first cell population containing lymphocytes, monocytes and basophils contained in the blood sample, on the basis of the forward scattered light and the side scattered light,
specifying a second cell population containing lymphocytes, monocytes and basophils, on the basis of the fluorescence from the third fluorescent label and the side scattered light,
identifying basophils in cells contained in both the specified first and second cell populations, on the basis of the fluorescences from the first and second fluorescent labels.

10. A method for counting basophils which comprises: (1) mixing and reacting a blood sample with an anti-CD123 antibody labeled with a first fluorescent label, an anti-CD294 antibody labeled with a second fluorescent label and an anti-CD45 antibody labeled with a third fluorescent label to prepare a measurement sample,
(2) introducing the measurement sample into a flow cell of a flow cytometer and irradiating, with light, cells in the measurement sample flowing in the flow cell,
(3) detecting fluorescences from the first, second and third fluorescent labels as well as side scattered light, emitted from the cells,
(4) identifying basophils on the basis of the detected fluorescences from the first, second and third fluorescent labels as well as the scattered light, and
(5) counting the identified basophils.

11. The method according to claim 10, wherein the scattered light is side scattered light.

12. The method according to claim 10, wherein the step (4) comprises:
specifying a cell population containing lymphocytes, monocytes and basophils, on the basis of the fluorescence from the third fluorescent label and the scattered light, and
identifying basophils in cells contained in the specified cell population, on the basis of the fluorescences from the first and second fluorescent labels.

13. The method according to claim 10, wherein the step (1) comprises treating, with a hemolyzing agent, the blood sample mixed and reacted with the antibodies, thereby lysing erythrocytes in the blood sample.

14. The method according to claim 13, wherein the hemolyzing agent is based on ammonium chloride.

15. The method according to claim 10, wherein the first, second and third fluorescent labels are fluorescent labels different from one another, which are selected from the group consisting of peridinin chlorophyll complex, fluorescein isothiocyanate, phycoerythrin, allophycocyanin, Texas Red and cy5.

16. A kit for measuring basophils which comprises:
a CD123 antibody labeled with a first fluorescent label, and a CD294 antibody labeled with a second fluorescent label.

17. The kit according to claim 16, which further comprises a CD45 antibody labeled with a third fluorescent label.

18. The kit according to claim 16, which further comprises a hemolyzing agent for lysing erythrocytes.

19. The kit according to claim 18, wherein the hemolyzing agent is based on ammonium chloride.
